(19) United States
(12) Patent Application Publication
(10) Pub. No.: US 2024/0379209 A1
(43) Pub. Date: Nov. 14, 2024

(54) AUTOMATED SCHEDULE GENERATION WITH CONTENT FILLING

(71) Applicant: Apple Inc., Cupertino, CA (US)

(72) Inventors: Udhyakumar NALLASAMY, San Jose, CA (US); Nicholas FOTI, Seattle, WA (US); Julie A. ARNEY, Los Gatos, CA (US); Cecilia A. COOPER, Santa Monica, CA (US); Lessa WAKUTSU, San Francisco, CA (US); Tahneetra CROSBY, Redondo Beach, CA (US); Sean W. JEWELL, Seattle, WA (US); Varun MAUDGALYA, Sunnyvale, CA (US); Troy J. CHEVALIER, Foster City, CA (US); Yujie LI, Kirkland, WA (US); Lisa A. WHEELER, Santa Monica, CA (US)

(21) Appl. No.: 18/237,875

(22) Filed: Aug. 24, 2023

Related U.S. Application Data
(60) Provisional application No. 63/465,221, filed on May 9, 2023.

(51) Int. Cl. G16H 20/30

(52) U.S. Cl. CPC G16H 20/30

(57) ABSTRACT

The subject system may be implemented by a processor circuit configured to receive a request for generating a workout schedule that includes one or more workout sessions and one or more workout content items associated with the one or more workout sessions. The request includes one or more workout session preferences and one or more workout content preferences. The processor circuit is configured to generate a schedule framework based on the one or more workout session preferences, the schedule framework comprising one or more scheduled workout sessions on one or more days of a week, generate the workout schedule by selecting, for each respective scheduled workout session in the schedule framework, a workout content item from a set of workout content items based at least in part on the one or more workout content preferences, and provide an indication of at least a portion of the workout schedule.

### CROSS-REFERENCE TO RELATED APPLICATIONS

The present application claims the benefit of U.S. Provisional Patent Application Ser. No. 63/465,221, entitled “AUTOMATED SCHEDULE GENERATION WITH CONTENT FILLING,” filed May 9, 2023, which is hereby incorporated herein by reference in its entirety and made part of the present U.S. Utility Patent Application for all purposes.

### TECHNICAL FIELD

The present description generally relates to scheduling content on electronic devices and, more particularly, to automated schedule generation with content filling.

### BACKGROUND

An electronic device such as a laptop, tablet, smartphone, or wearable device may generate a schedule according to predetermined parameters, such as those input by a user.

### BRIEF DESCRIPTION OF THE DRAWINGS

Certain features of the subject technology are set forth in the appended claims. However, for the purpose of explanation, several implementations of the subject technology are set forth in the following figures.

FIG. 1 illustrates an example network environment for automated schedule generation, in accordance with one or more implementations.

FIG. 2 depicts an example electronic device that may implement the subject methods and systems, in accordance with one or more implementations.

FIG. 3 depicts an example interface for inputting workout session preferences and/or workout content preferences, in accordance with one or more implementations.

FIG. 4 depicts example candidate daily schedules for a first active day in a week, in accordance with one or more implementations.

FIG. 5 depicts an example candidate schedule framework for selecting a candidate daily schedule for the first active day in the week, in accordance with one or more implementations.

FIG. 6 depicts an example process for content filling a schedule framework, in accordance with one or more implementations.

FIG. 7 depicts a flow diagram of an example process for automated schedule generation, in accordance with one or more implementations.

FIG. 8 depicts an example electronic system with which aspects of the present disclosure may be implemented, in accordance with one or more implementations.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Aspects of the subject technology provide a discrete optimization approach for generating personalized workout plans for users based on their input regarding their preferred workout days, workout duration, and workout types. The system accomplishes this in at least two phases: schedule framework generation and content filling.

In the schedule framework generation phase, the system uses discrete optimization techniques to generate a listing of workout session modalities and durations for designated days (e.g., active days) in a week (e.g., a calendar week). A user can select one or more days to workout (“active days”), a duration to workout on an active day, and one or more exercises (“modalities”). For each day in the week, the subject technology, via a greedy algorithm, may generate multiple candidate daily schedules for that particular day, where each daily schedule includes one or more workout sessions. For each generated candidate daily schedule, the subject technology, via a rollout algorithm, may generate multiple candidate weekly schedules (“schedule framework”) for the remaining active days in the week. For each generated candidate schedule framework, the subject technology may generate a score according to a set of predetermined rules (e.g., criteria/scores, such as 100 points for no repeat modalities in the same day, 80 points for no adjacent strength workouts, and the like). For that particular day, the subject technology may select the candidate daily schedule that generates the highest scoring schedule framework. The subject technology may repeat the process for each subsequent active day.

In the content filling phase, the subject technology may fill in content items (e.g., videos) for the workout sessions in the schedule framework according to workout content preferences (e.g., selected trainers and/or music genres). For each active day in the schedule framework and for each workout session in the active day, the subject technology may select workout content items (e.g., videos) whose modality and duration match the workout session. Each selected workout content item may be scored based on pre-defined parameters or criteria that are used to evaluate the quality and suitability of the video content for a given workout modality. The subject technology may then select a workout content item from the selected workout content items that maximizes a total content score for the active day and add the selected workout content item to the workout session

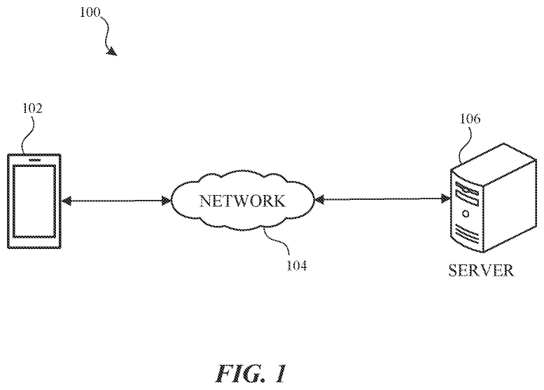

FIG. 1 illustrates an example network environment 100 for automated schedule generation, in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 may include an electronic device 102 and one or more servers (e.g., a server 106). The network 104 may communicatively (directly or indirectly) couple the electronic device 102 and the server 106. In one or more implementations, the network 104 may be an interconnected network of devices that may include, or may be communicatively coupled to, the Internet. For explanatory purposes, the network environment 100 is illustrated in FIG. 1 as including the electronic device 102 and the server 106; however, the network environment 100 may include any number of electronic devices and/or any number of servers communicatively coupled to each other directly or via the network 104.

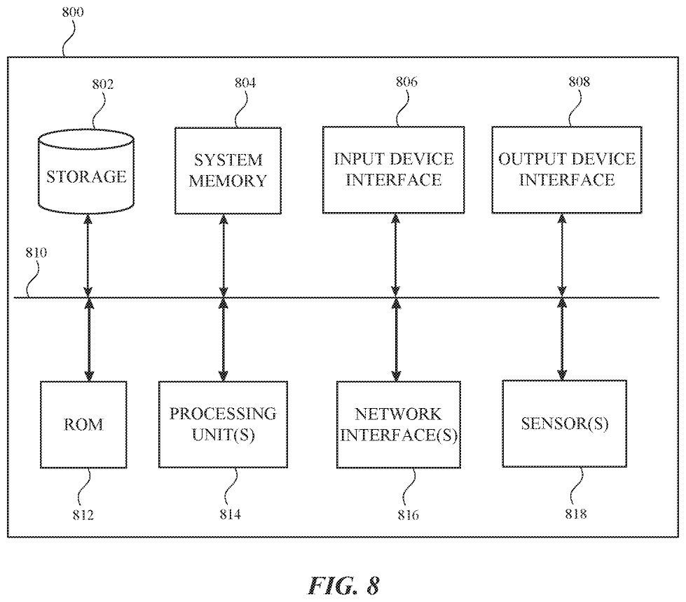

The electronic device 102 may be, for example, a wearable device such as a watch, a band, and the like, a desktop computer, a portable computing device such as a laptop computer, a smartphone, a peripheral device (e.g., a digital camera, headphones), a tablet device, or any other appropriate device that includes, for example, one or more wireless interfaces, such as WLAN radios, cellular radios, Bluetooth radios, Zigbee radios, near field communication (NFC) radios, and/or other wireless radios. In FIG. 1, by way of example, the electronic device 102 is depicted as a smartphone. The electronic device 102 may be, and/or may include all or part of, the electronic system discussed below with respect to FIG. 8. In one or more implementations, the electronic device 102 may include one or more sensors for generating motion data, biometric data, location data, and/or any other data that may be related to an exercise session (e.g., running).

In one or more implementations, one or more servers (e.g., the server 106) may store workout content items, which may be provided to and/or used by the electronic device 102. The workout content items may include audio, video, and/or media data and may correspond to a workout modality, duration, trainer, intensity, equipment, and/or the like. In some variations, the server 106 includes one or more app-specific modules (e g., plugins) that perform operations for a respective application (e.g., accessing workout content items).

In one or more implementations, the server 106 may store account information (e.g., user account, usernames/handles, or any other account-specific data) associated with the electronic device 102 and/or users thereof and/or users associated therewith. In one or more implementations, one or more servers (e.g., the server 106) may provide content (e.g., workout content items, application content, or any other suitable data) that is to be processed at the electronic device 102 by an application or operating system of the electronic device 102.

In one or more implementations, the electronic device 102 may be communicatively coupled to the server 106. The electronic device 102 be configured to communicate with the server 106 to send and/or receive, exercise data, workout schedules, workout session preferences, workout content preferences, schedule frameworks, location data, and/or any other information.

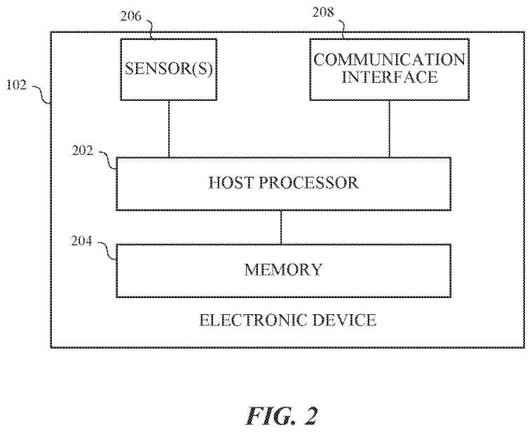

FIG. 2 depicts an example electronic device 102 that may implement the subject methods and systems, in accordance with one or more implementations. For explanatory purposes, FIG. 2 is primarily described herein with reference to the electronic device 102 of FIG. 1. However, this is merely illustrative, and features of the electronic device of FIG. 2 may be implemented in any other electronic device for implementing the subject technology. Not all of the depicted components may be used in all implementations, however, and one or more implementations may include additional or different components than those shown in FIG. 2. Variations in the arrangement and type of the components may be made without departing from the spirit or scope of the claims as set forth herein. Additional components, different components, or fewer components may be provided.

The electronic device 102 may include one or more of a host processor 202, a memory 204, one or more sensor(s) 206, and/or a communication interface 208. The host processor 202 may include suitable logic, circuitry, and/or code that enable processing data and/or controlling operations of the electronic device 102. In this regard, the host processor 202 may be enabled to provide control signals to various other components of the electronic device 102. The host processor 202 may also control transfers of data between various portions of the electronic device 102. The host processor 202 may further implement an operating system or may otherwise execute code to manage operations of the electronic device 102.

The memory 204 may include suitable logic, circuitry, and/or code that enable storage of various types of information such as received data, generated data, code, and/or configuration information. The memory 204 may include volatile memory (e.g., random access memory (RAM)) and/or non-volatile memory (e.g., read-only memory (ROM), flash, and/or magnetic storage). In one or more implementations, the memory 204 may store user location data, track data (e.g., lane information), exercise data (e.g., biometrics), account data, and any other data generated in the course of performing the processes described herein.

The sensor(s) 206 may include one or more motion sensors, biometric sensors, location sensors, and the like. The motion sensors may generate motion data based on the motion of the user (e.g., the user wearing the electronic device 102). The motion data may be used to determine, for example, the number of steps taken, distance traveled, and the like. The biometric sensors may generate biometric data based on, for example, the contact of the sensor with the user. For example, biometric data may include heart rate, respiratory rate, and the like.

The communication interface 208 may include suitable logic, circuitry, and/or code that enables wired or wireless communication, such as between the electronic device 102 and the server 106. The communication interface 208 may include, for example, one or more of a Bluetooth communication interface, an NFC interface, a Zigbee communication interface, a WLAN communication interface, a USB communication interface, a cellular interface, or generally any communication interface.

In one or more implementations, one or more of the host processor 202, the memory 204, the sensor(s) 206, the communication interface 208, and/or one or more portions thereof may be implemented in software (e.g., subroutines and code), may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

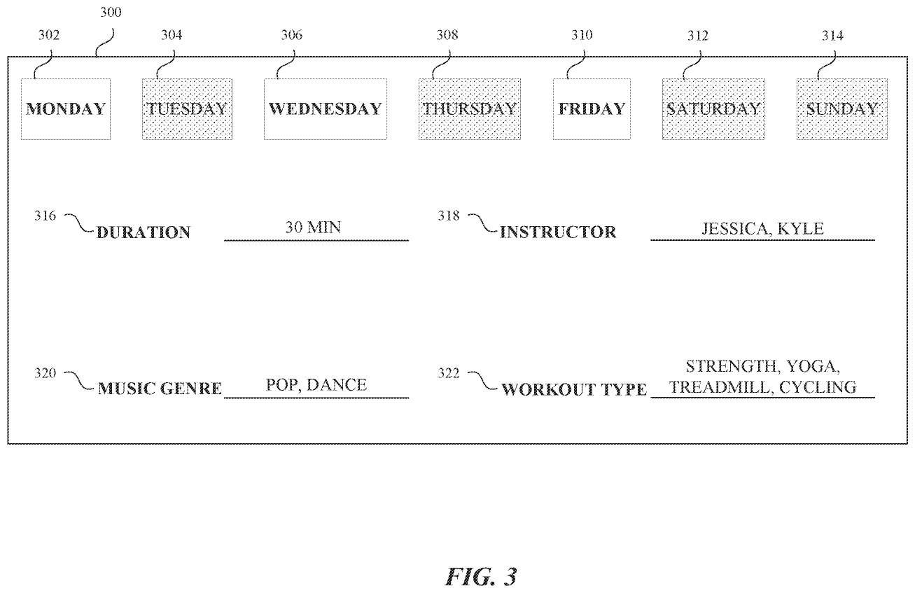

FIG. 3 depicts an example interface 300 for inputting workout session preferences and/or workout content preferences, in accordance with one or more implementations. The subject technology may receive one or more preferences from the user to generate a personalized workout schedule for the user. Interface 300 provides an example of how a user may input preferences and what preferences a user may input. The preferences may be included in a request to an electronic device (e.g., the electronic device 102) for generating a workout schedule. It should be understood that interface 300 is not intended to be limiting and other types of interfaces and preferences are contemplated.

Preferences may include workout session preferences, such as active days (e.g., days of the week in which to workout), modalities (e.g., workout types), daily duration (e.g., total duration of workout sessions in a daily schedule), and/or any other preferences relating to one or more workout sessions.

The interface 300 may include a button for each day of the week (e.g., Monday 302, Tuesday 304, Wednesday 306, Thursday 308, Friday 310, Saturday 312, and Sunday 314) that may be selected to indicate an active day that the user desires to workout. In the instance shown in FIG. 3, the workout schedule to be generated spans one week and the user indicates a desire to workout on Monday 302, Wednesday 306, and Friday 310 as the corresponding buttons are selected and the other buttons are shaded to indicate that they were not selected. Accordingly, the schedule generated may include workouts on Monday 302, Wednesday 306, and Friday 310.

The interface 300 may also include an input field 316 for the duration of a daily schedule. The input field 316 may receive as input a number indicating the number of minutes the daily schedule should be. For example, the input field 316 may include one or more durations (e.g., 10, 20, 30, 45, or 90 minutes) that may be selected by the user. In some implementations, the user may specify the duration of the daily schedule of each active day. In some implementations, the user may specify the number of weeks for which to generate a workout schedule. In the instance shown in FIG. 3, the user indicates a desire to workout 30 minutes per active day.

The interface 300 may also include an input field 322 for the modalities that may be included in the workout schedule. Modalities may include cycling, rowing, treadmill, high-intensity interval training (HIIT), kickboxing, dance, strength, Pilates, core, yoga, cooldown, meditation, and/or any other workout type. Modalities may be grouped into categories. For example, cardio may include cycling, rowing, treadmill, HIIT, kickboxing, and dance; strength may include strength, Pilates, and core; flexibility may include yoga and cooldown; and meditation may include meditation. The input field 322 may receive input text indicating the modalities that may be included in the workout schedule. For example, the input field 322 may be a dropdown menu including one or more modalities that may be selected by the user.

Preferences may also include workout content preferences, such as genre, instructor, popularity, recency, and/or any other preferences relating to the content provided to facilitate a workout session.

The interface 300 may include an input field 320 for the genre of the workout content items. The genre of a workout content item may include one or more styles for one or more aspects of the workout sessions and/or the workout content items. For example, the workout content item may be a video or an audio that includes music and the music may be categorized by one or more genres, such as pop, dance, and/or the like. In the interface 300, the input field 320 may be a dropdown menu including one or more genres that may be selected by the user.

The interface 300 may also include an input field 318 for the instructor of the workout content item. The workout content item may include one or more individuals that facilitate one or more aspects of the workout session and/or the workout content item. For example, a video of a workout session may be led by an instructor, and the instructor may be accompanied by one or more other instructors to perform one or more modified versions of the workout session. In the interface 300, the input field 318 may be a dropdown menu including one or more instructors that may be selected by the user.

In some implementations, the options available for input via the input fields (e.g., input fields 316-322) may be dependent on one or more other input fields. For example, if Jessica and Kyle are selected instructors in input field 318 and they only instruct in workout content items associated with the yoga and strength modalities, respectively, then the treadmill and cycling options in the input field 322 may not be selectable by the user.

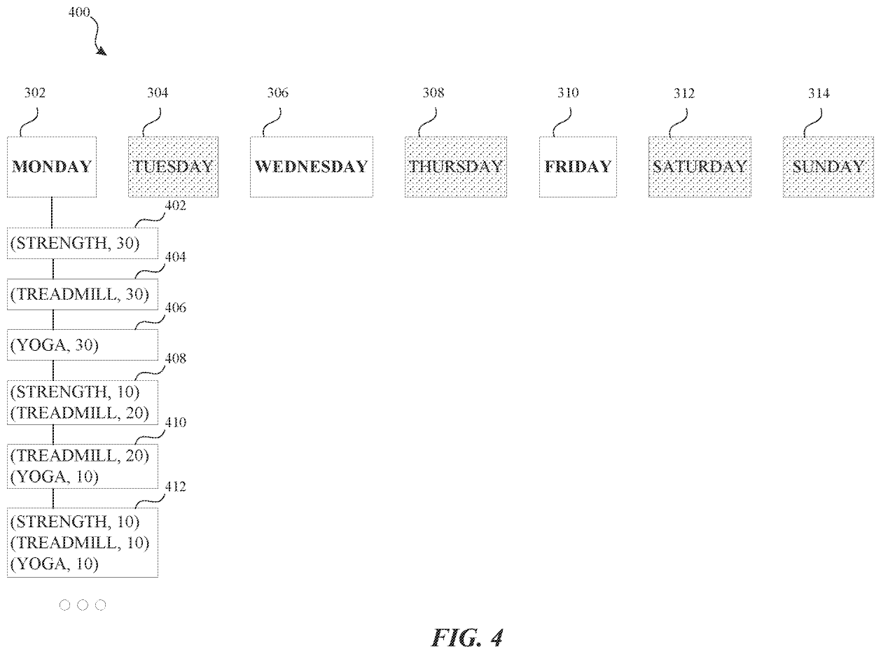

FIG. 4 depicts example candidate daily schedules 402-412 for a first active day (e.g., Monday 302) in the week 400, in accordance with one or more implementations. Once a request for generating a workout schedule, the system (e.g., the electronic device 102) may generate a personalized workout schedule for the user. The workout schedule framework may include one or more scheduled workout sessions on one or more days of the week 400 and may be generated based on one or more workout session preferences.

To generate the schedule framework, the system may utilize a greedy algorithm for generating and selecting daily schedules for each active day in the week 400 (e.g., Monday 302, Wednesday 306, and Friday 310).

As shown in FIG. 4, the system considers candidate daily schedules that could be utilized for the first active day (e g., Monday 302). For example, the system generates candidate daily schedules 402-412 for Monday 302. The candidate daily schedules 402-412 may be generated by enumerating combinations of workout session preferences, such as modality and duration. It should be understood that although six candidate daily schedules are depicted, the number of candidate daily schedules generated are not limited to six.

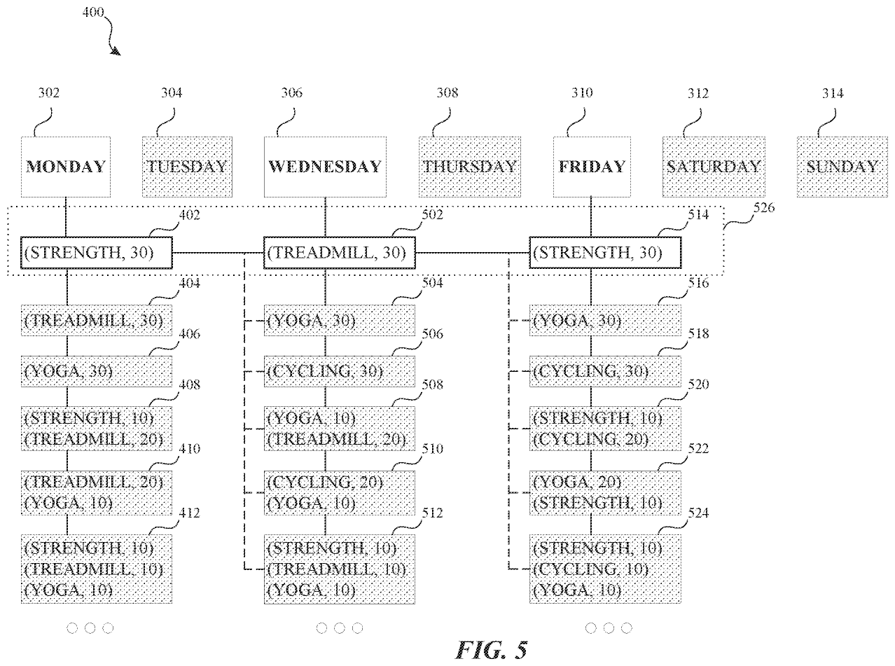

FIG. 5 depicts an example candidate schedule framework 526 for selecting a candidate daily schedule for the first active day (e.g., Monday 302) in the week 400, in accordance with one or more implementations. For each candidate daily schedule, the system utilizes a rollout algorithm to generate candidate schedule frameworks (the “rollouts”) for the week 400. The system selects the candidate daily schedule for the first active day that corresponds to the highest scoring, valid candidate schedule framework. This process is repeated for each subsequent active day (e.g., generating rollouts and selecting a daily schedule for each subsequent active day) until the week 400 is complete. The system may repeat the greedy and rollout algorithms for a specified number of weeks.

To illustrate, continuing from FIG. 4, each candidate daily schedule 402-412 may be utilized as the basis for generating one or more candidate schedule frameworks to determine which candidate daily schedule 402-412 may be selected for the final schedule framework. As shown in FIG. 5, the system may first analyze a candidate daily schedule 402 for Monday 302. With the candidate daily schedule 402, the system may generate one or more candidate daily schedules 502-512 for Wednesday 306 (e.g., the next active day). The candidate daily schedules 502-512 may be generated in a manner similar to the candidate daily schedules 402-412. The system may also generate candidate daily schedules for Friday 310 corresponding to each candidate daily schedule 502-512. For candidate daily schedule 502, for example, the system may generate candidate daily schedules 514-524. The candidate daily schedules 514-524 may be generated in a manner similar to the candidate daily schedules 402-412 and/or candidate daily schedules 514-524.

This process may be repeated for each candidate daily schedule 402-412 of Monday 302. For example, the system may move to the candidate daily schedule 404 for Monday 302. With the candidate daily schedule 404, the system may generate candidate daily schedules for Wednesday 306, which may be different than the candidate daily schedules 502-512. The system may also generate candidate daily schedules for Friday 310 corresponding to each candidate daily schedule of Wednesday 306, which may be different than the candidate daily schedules 514-524.

Once the candidate schedule frameworks are generated, the system may score each candidate schedule framework (e.g., the candidate schedule framework 526) based on one or more predetermined schedule framework criteria. These criteria may include avoiding repeated modalities on the same day, avoiding strength workouts on a day that follows a strength workout, avoiding modalities of the same category on the same day, alternating modalities within categories on subsequent days, and/or the like. Each criterion may be assigned a weight and/or score, and the score of a candidate schedule framework may then be calculated as the sum of its scores for each criterion. This way the candidate schedule frameworks are scored such that candidate schedule frameworks with a desired structure have higher scores than those with an undesired structure, where desirability may be in terms of fitness goals, efficient fitness training, and/or the like.

A desired schedule framework might include a mix of cardio, strength, and flexibility workouts throughout the week, with no more than two workouts of the same modality on the same day and at least one rest day per week. An undesired schedule framework, on the other hand, might include too many strength workouts in a row, or too many workouts of the same category in a day, making it less desirable for achieving fitness goals or efficient training. A candidate schedule framework might exhibit a combination of desired and undesired characteristics and thus both awarded points and penalized points. The system scores each candidate schedule framework based on how well it satisfies these criteria, awarding higher scores to those that exhibit more desired characteristics and penalizing those that exhibit more undesired characteristics. Ultimately, the system recommends the candidate schedule framework with the highest score as the most suitable schedule for the user.

For example, the system may award 100 points to a candidate schedule framework that includes all specified modalities in the plan. Similarly, 100 points may be given to a schedule framework that avoids repeating modalities on the same day, while a penalty may be applied to those that repeat a modality in a day. Additionally, the system may apply a penalty of 80 points to a candidate schedule framework that includes adjacent strength workouts, and the penalty may also consider the preceding and following days. Another penalty of 80 points may be applied to candidate schedule frameworks that include a combination of yoga and mindful cooldown modalities in the same day. The system may also discourage the repetition of workouts of the same category within a day and look to encourage days that contain workouts from different categories, which may be assigned a point value of 50. Moreover, the system may encourage alternating modalities within categories on subsequent days, which may be assigned a score of 50. Finally, the system may discourage repeating modalities in the same category across days and instead encourage alternating modalities from the same category, with a point value of 50.

Once the candidate schedule frameworks are scored, the system may select the candidate daily schedule associated with the highest scoring candidate schedule framework from the candidate daily schedules 402-412 for Monday 302. For example, the schedule framework 526 has the highest score for the candidate schedule frameworks generated from candidate daily schedules 402-412, and thus the candidate daily schedule 402 may be the selected daily schedule for Monday 302.

Once the daily schedule 402 is selected for Monday 302, the system may proceed to Wednesday 306 (e g., the next active day). The system may generate one or more candidate daily schedules for Wednesday 306 and perform the greedy and rollout algorithms for the rest of the week 400 based on the generated candidate daily schedules. For example, in performing the greedy and rollout algorithms, Monday 302 may remain constant because its daily schedule has already been selected, while candidate daily schedules are generated for Wednesday 306 and Friday 310. The candidate schedule frameworks may be scored, and the candidate daily schedule associated with the highest scoring candidate schedule framework may be selected for Wednesday 306.

Once a daily schedule is selected for Monday 302 and Wednesday 306, the system may proceed to Friday 310 (e.g., the next active day). The system may generate one or more candidate daily schedules for Friday 310. Each candidate daily schedule of Friday 310, along with the selected daily schedules for Monday 302 and Wednesday 306, may be considered a candidate schedule framework because Friday 310 is the last active day of the week 400 and the only remaining active day without a determined daily schedule. The candidate schedule frameworks may be scored, and the candidate daily schedule associated with the highest scoring candidate schedule framework may be selected for Friday 310.

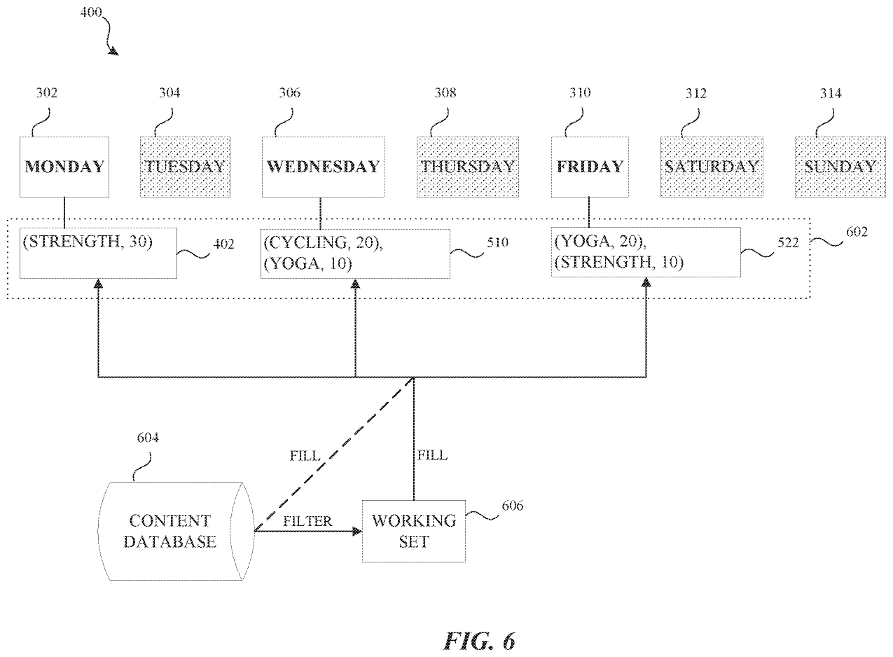

FIG. 6 depicts an example process for content filling a schedule framework 602, in accordance with one or more implementations. After generating the schedule framework 602 as shown in FIG. 4 and FIG. 5, the system may curate workout content items (e.g., audio, video, and/or other media) for the schedule framework 602.

The system may access workout content items from a database 604, which may be local (e.g., on the electronic device 102) and/or remote (e.g., on the server 106). In some implementations, the system may generate a working set 606 of workout content items by querying the database 604 for only the workout content items whose modality and duration are included in the schedule framework 602.

With the workout content items (e.g., from the content database 604 and/or the working set 606), the system may iterate through the workout sessions in the schedule framework scoring the workout content items and selecting the highest scoring workout content item for each workout session. For example, the system may iterate through the workout sessions in chronological order, filling each workout session with a workout content item that has been scored and selected by the system.

The system may score one or more workout content items based on a set of predetermined content criteria. The predetermined content criteria may include scores associated with characteristics (e.g., relevance, recency, popularity, and the like) of the content item to evaluate the suitability of the workout content item for the schedule framework and the user's content preferences.

For example, one criterion may be to discourage videos that the user has watched before, which may be assigned a score of 50. This may help expose users to new and diverse content, which can help them maintain their interest and motivation. Another criterion may be content recency, which may be assigned a score of 10. This criterion may prefer more recent content over older content, with a preference for content released this week over content from the previous week, and so on. This may help keep content up-to-date and relevant to the user's goals and preferences. Another criterion may be to avoid scheduling two videos with similar content or style in consecutive workouts, which may be assigned a score of 20. This may help keep the workouts fresh and engaging. The user's trainer and genre preferences may also be taken into account, with a score of 20 and 10 respectively. Videos that feature trainers or music genres that the user has indicated a preference for may receive a higher score. The boost in score may be determined by the number of overlapping trainers and music genres that the video has with the user's specified preferences.

The predetermined content criteria may include scores associated with characteristics such as relevance, recency, popularity, and the like, to evaluate the suitability of the workout content item for the schedule framework and the user's content preferences. For example, a workout content item may receive points for being recent, associated with a genre preferred by the user, and/or associated with an instructor preferred by the user. However, it may lose points for being previously watched and/or following a workout session having the same category or modality. The content score consists of a number of terms with weights according to their importance. The terms include discouraging videos the user has watched before (50 points), preferring more recent content over older content (10 points), avoiding repeating videos back-to-back (20 points), boosting videos with the trainers and music genres that the user indicated they prefer (20 and 10 points respectively), and encouraging selecting videos from a diverse set of trainers (1 and 0 points respectively). The boost for trainer and genre preferences is determined by the number of overlapping trainers and music genres the video has with the user's specified preferences. Ultimately, the workout content item with the greatest number of and/or highest weighted desired characteristics may be awarded a higher score than a workout content item with fewer and/or lower weighted desired characteristics.

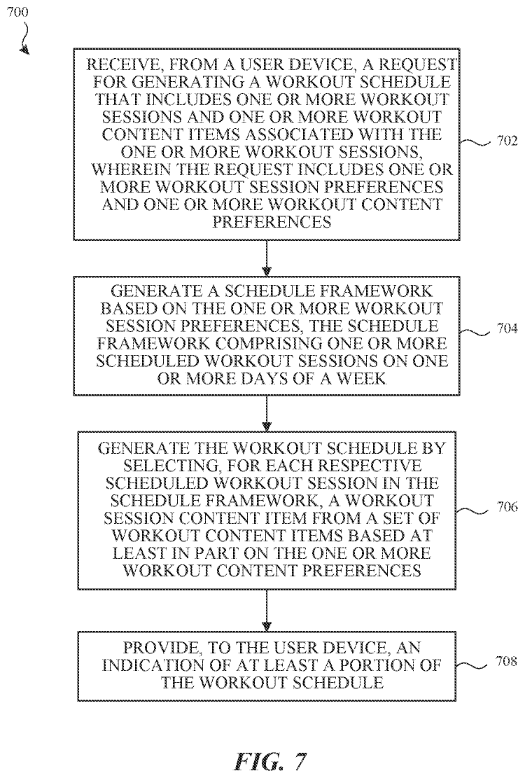

FIG. 7 depicts a flow diagram of an example process 700 for automated schedule generation, in accordance with one or more implementations. For explanatory purposes, the process 700 is primarily described herein with reference to the electronic device 102 of FIG. 1. However, the process 700 is not limited to the electronic device 102, and one or more blocks of the process 700 may be performed by one or more other components of the electronic device 102, and/or by other suitable devices (e.g., the server 106). Further, for explanatory purposes, the blocks of the process 700 are described herein as occurring sequentially or linearly. However, multiple blocks of the process 700 may occur in parallel. In addition, the blocks of the process 700 need not be performed in the order shown and/or one or more blocks of the process 700 need not be performed and/or can be replaced by other operations.

At block 702, the electronic device 102 may receive a request for generating a workout schedule. The workout schedule may include one or more workout sessions and one or more workout content items associated with the one or more workout sessions. A workout session may be a scheduled period (e.g., associated with a day and/or time) in which a user desires to workout, and a workout content item may be audio, video, or any other media used to facilitate the workout (e.g., a guided exercise video).

The request may include one or more workout session preferences and/or one or more workout content preferences. Workout session preferences may include preferences relating to the workout session, such as active days (e.g., days in which to workout), modalities (e.g., types of exercises), and duration (e.g., how long to workout per active day), and workout content preferences may include preferences relating to the content presented in association with a workout session, such as genre (e.g., the music/style of the content item), instructor (e.g., the instructor featured in a content item), popularity (e.g., number of views of the content item among other users), and recency (e.g., the age of the content item).

In some implementations, the request may also include a desired number of weeks for which to generate a workout schedule.

In some implementations, the request may be received from an application on the electronic device 102. For example, the interface 300 may include a form for the user to select active days and input workout session preferences and workout content item preferences, and a request may be generated when the form is submitted.

In some implementations, the electronic device 102 may receive the request from another device and generate the workout schedule for the other device.

At block 704, the electronic device 102 may generate a schedule framework (e.g., a schedule framework 602) based on the one or more workout session preferences. The schedule framework may include one or more workout sessions scheduled for one or more active days of a set of days (e.g., a week). In some implementations, the schedule framework may include one or more workout sessions scheduled for particular times of an active day.

To generate the schedule framework, the electronic device 102 may iterate through the active days to determine a daily schedule (e.g., one or more modalities and corresponding duration) for each respective active day.

To determine a daily schedule for each respective active day, the electronic device 102 may generate one or more candidate daily schedules (e.g., sets of workout sessions) for the active day, rollout the candidate daily schedules through the remaining active days by generating one or more candidate schedule frameworks, score the candidate schedule frameworks, and select the candidate daily schedule (for the active day) associated with the highest scoring candidate schedule framework.

The electronic device 102 may generate one or more candidate daily schedules for the first active day (e.g., Monday 302). The generated candidate daily schedules may be combinations of workout session preferences, such as modality and duration. For example, if the workout session preferences indicate a preference for a 90-minute duration and cycling, rowing, and strength, then the daily schedules may include one 45-minute cycling session and one 45-minute strength session, two 30-minute strength sessions (e.g., upper body and lower body) and one 30-minute rowing session, and so on.

The electronic device 102 may also generate, via a rollout algorithm, one or more candidate schedule frameworks for each respective candidate daily schedule of the first active day. To generate the candidate schedule frameworks, for each respective candidate daily schedule of the first active day (e.g., Monday 302), the electronic device 102 may generate one or more candidate daily schedules for the second active day (e.g., Wednesday 306); for each respective candidate daily schedule of the second active day, the electronic device 102 may also generate one or more candidate daily schedules for the third active day (e.g., Friday 310); and so on.

The electronic device 102 may generate a score for each respective schedule framework. The score may be based on a set of predetermined schedule framework criteria that may add points to promote desired schedule framework characteristics and/or remove points to penalize undesired schedule framework characteristics. The predetermined schedule framework criteria may include intensity. For example, the schedule framework may include a maximum of three HIIT workout sessions. The predetermined schedule framework criteria may also include modality balance. For example, a penalty may apply when scheduling workout sessions having a treadmill modality on two consecutive days when there is another workout session option having a modality in the same category (e.g., cycling is also in the cardio category and may be a preferred modality to follow a day that includes treadmill). The predetermined schedule framework criteria may also include workout session variety. For example, points may be added to schedule frameworks that include multiple workout sessions for the target duration over a single workout session. The predetermined schedule framework criteria may further include equipment availability. For example, a schedule framework may be penalized for requiring multiple sets of equipment in a single daily schedule, such as treadmill, dumbbells, and yoga mat. Other schedule framework criteria are contemplated.

The electronic device 102 may select the daily schedule associated with the highest scoring candidate schedule framework for the first active day. For each remaining active day in the week 400, the electronic device 102 may generate one or more candidate daily schedules for the active day, rollout the candidate daily schedules through the remaining active days, score the candidate schedule frameworks, and select the candidate daily schedule (for the active day) associated with the highest scoring candidate schedule framework.

In some implementations, the electronic device 102 may also rearrange the scheduled workout sessions in one or more active days of the schedule framework. Workout sessions may be rearranged so that active days may end with particular modalities when present. For example, if a schedule framework includes yoga, the workout sessions in each daily schedule in the schedule framework may be rearranged such that yoga concludes the respective daily schedule. Workout sessions may also or instead be rearranged to promote consistency of the workout session modality order across days. For example, workout sessions may be arranged by category and by modality within a category as shown in the following exemplary table where the top row indicates category, categories are ordered from left to right, and modalities are ordered from top to bottom:

| Cardio | Strength | Flexibility | Meditation |
|---|---|---|---|
| Cycling | Strength | Yoga | Meditation |
| Rowing | Pilates | Mindful Cooldown | |
| Treadmill | Core | | |
| HIIT | | | |
| Kickboxing | | | |
| Dance | | | |

At block 706, the electronic device 102 may generate the workout schedule by curating content for each scheduled workout session in the schedule framework generated at block 704. For each respective scheduled workout session in the schedule framework, the electronic device 102 may select a workout content item from a set of workout content items based at least in part on the user's workout content preferences (e.g., from the request for generating a workout schedule).

The set of workout content items may include video, audio, or other media items that may be stored in locally (e.g., on the electronic device 102) and/or remotely (e.g., on the server 106). The set of workout content items may be selected directly from a complete repository of workout content items (e.g., the content database 604). The set of workout content items may also or instead be selected from a working set of workout session content (e.g., the working set 606), which includes a set of workout content items from the complete repository of workout content items that are filtered by workout session modality and/or workout session length in order to reduce the number of workout content items considered when curating content for the schedule framework. For example, considering a 30 minute daily schedule including a cycling modality for 20 minutes and a yoga modality for 10 minutes, a working set of workout content may include videos that are 20 minute cycling videos and 10 minute yoga videos.

With the set of workout content items, the electronic device 102 may iterate through the workout sessions in the schedule framework scoring the workout content items and selecting the highest scoring workout content item for each workout session. For example, the electronic device 102 may iterate through the workout sessions in chronological order, filling each workout session with a workout content item that has been scored and selected by the electronic device 102.

The electronic device 102 may score one or more workout content items based on a set of predetermined content criteria. The predetermined content criteria may include scores associated the relevance (e.g., how related a content item is to content items that the user has previously seen), recency (e.g., how new the content item is), popularity (e.g., how much the content item has been viewed by others), and/or the like, of the workout content item to evaluate the suitability of the workout content item for the schedule framework and the user's content preferences. For example, a workout content item may receive points for being recent, associated with a genre preferred by the user, and/or associated with an instructor preferred by the user but may lose points for being previously watched and/or following a workout session having the modality or modality in the same category. In some implementations, the predetermined content criteria may also include modality-specific rules. For example, different types of workout sessions in the strength modality (e.g., total body, lower body, and upper body) may be treated as different modalities for purposes of content curation. In some implementations, selecting the workout content item for a scheduled workout session may correspond to at least one other selected session content item.

In some implementations, a user may request that a workout schedule be generated for multiple weeks. To do so, the electronic device 102 may generate a new workout schedule for each week for the number of desired weeks. For example, the electronic device 102 may repeat block 704 and block 706 for the desired number of weeks. In some implementations, the workout schedule may be generated based on the previous week's workout schedule For example, the user may indicate a preference for alternating between strength-focused weeks and cardio-focused weeks, in which case the electronic device 102 may consider the workout sessions of the previous week's workout schedule in generating the workout schedule for the target week.

At block 708, the electronic device 102 may output, send, save, transfer, display, or otherwise provide an indication of at least a portion of the workout schedule. The indication may include a link, notification, data structure, or any other indicator of the workout schedule or portion thereof.

FIG. 8 depicts an example electronic system 800 with which aspects of the present disclosure may be implemented, in accordance with one or more implementations. The electronic system 800 can be, and/or can be a part of, any electronic device for generating the features and processes described in reference to FIGS. 1-7, including but not limited to a laptop computer, tablet computer, smartphone, and wearable device (e.g., smartwatch, fitness band). The electronic system 800 may include various types of computer-readable media and interfaces for various other types of computer-readable media. The electronic system 800 includes one or more processing unit(s) 814, a persistent storage device 802, a system memory 804 (and/or buffer), an input device interface 806, an output device interface 808, a bus 810, a ROM 812, one or more processing unit(s) 814, one or more network interface(s) 816, one or more sensors 818, and/or subsets and variations thereof.

The bus 810 collectively represents all system, peripheral, and chipset buses that communicatively connect the numerous internal devices of the electronic system 800. In one or more implementations, the bus 810 communicatively connects the one or more processing unit(s) 814 with the ROM 812, the system memory 804, and the persistent storage device 802. From these various memory units, the one or more processing unit(s) 814 retrieves instructions to execute and data to process in order to execute the processes of the subject disclosure. The one or more processing unit(s) 814 can be a single processor or a multi-core processor in different implementations.

The ROM 812 stores static data and instructions that are needed by the one or more processing unit(s) 814 and other modules of the electronic system 800. The persistent storage device 802, on the other hand, may be a read-and-write memory device The persistent storage device 802 may be a non-volatile memory unit that stores instructions and data even when the electronic system 800 is off. In one or more implementations, a mass-storage device (such as a magnetic or optical disk and its corresponding disk drive) may be used as the persistent storage device 802.

In one or more implementations, a removable storage device (such as a floppy disk, flash drive, and its corresponding disk drive) may be used as the persistent storage device 802. Like the persistent storage device 802, the system memory 804 may be a read-and-write memory device. However, unlike the persistent storage device 802, the system memory 804 may be a volatile read-and-write memory, such as RAM. The system memory 804 may store any of the instructions and data that one or more processing unit(s) 814 may need at runtime. In one or more implementations, the processes of the subject disclosure are stored in the system memory 804, the persistent storage device 802, and/or the ROM 812. From these various memory units, the one or more processing unit(s) 814 retrieves instructions to execute and data to process in order to execute the processes of one or more implementations.

The bus 810 also connects to the input device interfaces 806 and output device interfaces 808. The input device interface 806 enables a user to communicate information and select commands to the electronic system 800. Input devices that may be used with the input device interface 806 may include, for example, alphanumeric keyboards, touch screens, and pointing devices. The output device interface 808 may enable the electronic system 800 to communicate information to users. For example, the output device interface 808 may provide the display of images generated by electronic system 800. Output devices that may be used with the output device interface 808 may include, for example, printers and display devices, such as a liquid crystal display (LCD), a light emitting diode (LED) display, an organic light emitting diode (OLED) display, a flexible display, a flat panel display, a solid-state display, a projector, or any other device for outputting information.

One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

The bus 810 also connects to sensor(s) 818. The sensor(s) 818 may include a location sensor, which may be used in determining device position based on positioning technology For example, the location sensor may provide for one or more of GNSS positioning, wireless access point positioning, cellular phone signal positioning, Bluetooth signal positioning, image recognition positioning, and/or an INS (e.g., via motion sensors such as an accelerometer and/or gyroscope). In one or more implementations, the sensor(s) 818 may be utilized to detect movement, travel and orientation of the electronic system 800. For example, the sensor(s) may include an accelerometer, a rate gyroscope, and/or other motion-based sensor(s). The sensor(s) 818 may include one or more biometric sensors for determining the exercise data of a user such as heart rate, respiratory rate, and any other biometric data.

Finally, as shown in FIG. 8, the bus 810 also couples the electronic system 800 to one or more networks and/or to one or more network nodes through the one or more network interface(s) 816. In this manner, the electronic system 800 can be a part of a network of computers (such as a local area network, a wide area network, an Intranet, or a network of networks, such as the Internet). Any or all components of the electronic system 800 can be used in conjunction with the subject disclosure.

Implementations within the scope of the present disclosure can be partially or entirely realized using a tangible computer-readable storage medium (or multiple tangible computer-readable storage media of one or more types) encoding one or more computer-readable instructions The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessors or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station,” “receiver,” “computer,” “server,” “processor,” and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refers to only A, only B, or only C, any combination of A, B, and C; and/or at least one of each of A, B, and C.

The predicate words “configured to,” “operable to,” and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, one or more implementations, one or more implementations, an embodiment, the embodiment, another embodiment, one or more implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration.” Any implementation described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The previous description is provided to enable any person skilled in the art to practice the various aspects described herein Various modifications to these aspects will be readily apparent to those skilled in the art, and the generic principles defined herein may be applied to other aspects. Thus, the claims are not intended to be limited to the aspects shown herein but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

As described above, one aspect of the present technology is the gathering and use of data available from specific and legitimate sources for file sharing. The present disclosure contemplates that in some instances, this gathered data may include personal information data that uniquely identifies or can be used to identify a specific person. Such personal information data can include demographic data, location-based data, online identifiers, telephone numbers, email addresses, home addresses, images, videos, audio data, data or records relating to a user's health or level of fitness (e.g., vital signs measurements, medication information, exercise information), date of birth, or any other personal information.

The present disclosure recognizes that the use of such personal information data, in the present technology, can be used to the benefit of users. For example, personal information data can be used for file sharing. Accordingly, the use of such personal information data may facilitate transactions (e.g., online transactions). Further, other uses for personal information data that benefit the user are also contemplated by the present disclosure. For instance, health and fitness data may be used, in accordance with the user's preferences to provide insights into their general wellness or may be used as positive feedback to individuals using technology to pursue wellness goals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates implementations in which users selectively block the use of, or access to, personal information data. That is, the present disclosure contemplates that hardware and/or software elements can be provided to prevent or block access to such personal information data. For example, in the case of file sharing, the present technology can be configured to allow users to select to “opt-in” or “opt-out” of participation in the collection of personal information data during registration for services or anytime thereafter. In addition to providing “opt-in” and “opt-out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health-related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed implementations, the present disclosure also contemplates that the various implementations can also be implemented without the need for accessing such personal information data. That is, the various implementations of the present technology are not rendered inoperable due to the lack of all or a portion of such personal information data.

What is claimed is:

1. A method comprising:
receiving, from a user device, a request for generating a workout schedule that includes one or more workout sessions and one or more workout content items associated with the one or more workout sessions, wherein the request includes one or more workout session preferences and one or more workout content preferences;
generating a schedule framework based on the one or more workout session preferences, the schedule framework comprising one or more scheduled workout sessions on one or more days of a week;
generating the workout schedule by selecting, for each respective scheduled workout session in the schedule framework, a workout content item from a set of workout content items based at least in part on the one or more workout content preferences; and
providing, to the user device, an indication of at least a portion of the workout schedule.

2. The method of claim 1, wherein generating the schedule framework comprises, for each respective day of the one or more days of the schedule framework:
generating one or more sets of workout sessions for a respective day of the schedule framework;
generating, for each respective set of workout sessions, one or more candidate schedule frameworks;
scoring each respective candidate schedule frameworks of the one or more candidate schedule frameworks based on a set of predetermined schedule framework criteria; and
selecting, for the respective day of the schedule framework, a respective set of workout sessions associated with a respective candidate schedule framework having a highest score among the one or more candidate schedule frameworks.

3. The method of claim 2, wherein the predetermined schedule framework criteria comprise intensity, modality balance, variety, and equipment availability.

4. The method of claim 1, further comprising, before selecting the workout content item:
filtering the set of workout content items based on the one or more scheduled workout sessions in the schedule framework.

5. The method of claim 1, wherein selecting the workout content item comprises, for each respective scheduled workout session in the schedule framework:
scoring, for each respective workout content item in the set of workout content items, a respective workout content item based on a set of predetermined content criteria; and
selecting, for a respective scheduled workout session, a workout content item having a highest score from among the scored set of workout content items.

6. The method of claim 5, wherein the predetermined content criteria comprise relevance, recency, popularity, and the workout content preferences.

7. The method of claim 1, wherein the workout session preferences comprise active days, modalities, and duration.

8. The method of claim 1, wherein the workout content preferences comprise genre, instructor, popularity, and recency.

9. The method of claim 1, wherein the request further also includes a number of weeks, and the schedule framework spans one week.

10. The method of claim 9, further comprising:
repeating the receiving, generating, selecting, and providing for the number of weeks such that each week corresponds to a respective schedule framework.

11. The method of claim 1, wherein selecting the workout content item for a respective scheduled workout session corresponds to at least one other selected workout content item.

12. The method of claim 1, further comprising:
rearranging, for each respective day in the schedule framework, one or more scheduled workout sessions associated with the respective day such that the one or more scheduled workout sessions are in a predetermined order.

13. An electronic device comprising:
a memory; and
a processor circuit configured to:
receive, from a user device, a request for generating a workout schedule that includes one or more workout sessions and one or more workout content items associated with the one or more workout sessions, wherein the request includes one or more workout session preferences and one or more workout content preferences;
generate a schedule framework based on the one or more workout session preferences, the schedule framework comprising one or more scheduled workout sessions on one or more days of a week;
generate the workout schedule by selecting, for each respective scheduled workout session in the schedule framework, a workout content item from a set of workout content items based at least in part on the one or more workout content preferences; and
provide, to the user device, an indication of at least a portion of the workout schedule.

14. The electronic device of claim 13, wherein generating the schedule framework comprises, for each respective day of the one or more days of the schedule framework:
generating one or more sets of workout sessions for a respective day of the schedule framework;
generating, for each respective set of workout sessions, one or more candidate schedule frameworks;
scoring each respective candidate schedule frameworks of the one or more candidate schedule frameworks based on a set of predetermined schedule framework criteria; and
selecting, for the respective day of the schedule framework, a respective set of workout sessions associated with a respective candidate schedule framework having a highest score among the one or more candidate schedule frameworks.

15. The electronic device of claim 13, wherein the processor circuit is further configured to, before selecting the workout content item:
filter the set of workout content items based on the one or more scheduled workout sessions in the schedule framework.

16. The electronic device of claim 13, wherein selecting the workout content item comprises, for each respective scheduled workout session in the schedule framework:
scoring, for each respective workout content item in the set of workout content items, a respective workout content item based on a set of predetermined content criteria; and
selecting, for a respective scheduled workout session, a workout content item having a highest score from among the scored set of workout content items.

17. The electronic device of claim 13, wherein selecting the workout content item for a respective scheduled workout session corresponds to at least one other selected workout content item.

18. A non-transitory computer-readable medium comprising:
computer-readable instructions that, when executed by a processor, cause the processor to perform one or more operations comprising:
receiving, from a user device, a request for generating a workout schedule that includes one or more workout sessions and one or more workout content items associated with the one or more workout sessions, wherein the request includes one or more workout session preferences and one or more workout content preferences;
generating a schedule framework based on the one or more workout session preferences, the schedule framework comprising one or more scheduled workout sessions on one or more days of a week;
generating the workout schedule by selecting, for each respective scheduled workout session in the schedule framework, a workout content item from a set of workout content items based at least in part on the one or more workout content preferences; and
providing, to the user device, an indication of at least a portion of the workout schedule.

19. The non-transitory computer-readable medium of claim 18, wherein generating the schedule framework comprises, for each respective day of the one or more days of the schedule framework:
generating one or more sets of workout sessions for a respective day of the schedule framework;
generating, for each respective set of workout sessions, one or more candidate schedule frameworks;
scoring each respective candidate schedule frameworks of the one or more candidate schedule frameworks based on a set of predetermined schedule framework criteria; and
selecting, for the respective day of the schedule framework, a respective set of workout sessions associated with a respective candidate schedule framework having a highest score among the one or more candidate schedule frameworks.

20. The non-transitory computer-readable medium of claim 18, wherein selecting the workout content item comprises, for each respective scheduled workout session in the schedule framework:
scoring, for each respective workout content item in the set of workout content items, a respective workout content item based on a set of predetermined content criteria; and
selecting, for a respective scheduled workout session, a workout content item having a highest score from among the scored set of workout content items.
